(19) United States
(12) Patent Application Publication
(10) Pub. No.: US 2006/0229663 A1
(43) Pub. Date: Oct. 12, 2006

(54) Breast and subcutaneous surgery hemostasis apparatus

(76) Inventors: Kwok-Wai Chiu, Plano, TX (US); Lee-Ting Chiu, Plano, TX (US)

Correspondence Address: BIRCH STEWART KOLASCH & BIRCH, PO BOX 747, FALLS CHURCH, VA, 22040-0747, US

(21) Appl. No.: 11/102,785

(22) Filed: Apr. 11, 2005

(51) Int. Cl. A61B 17/00

(52) U.S. Cl. 606/201

(57) ABSTRACT

A hemostasis device for controlling bleeding during a surgery is provided. The hemostasis device includes a first portion, which has a close structure, an adjusting device connected with the first portion, and a second portion connected with the adjusting device. The first portion is affixed to the patient to surround the surgical area, and a user adjusts the adjusting device to move the second portion to stress the patient to control bleeding during the surgery.

### FIELD OF THE INVENTION

The present invention relates to a medical instrument, in general, and to a hemostasis apparatus for preventing profuse bleeding, particularly during a surgical procedure.

### BACKGROUND OF THE INVENTION

In performing many medical procedures, it is necessary to make incisions. It is just as necessary to properly close the incision. Physicians would have experienced profuse bleeding while performing cutaneous or subcutaneous surgery on a patient. The profuse bleeding will cause numerous complications that will raise the risk of the surgery.

In prior instances, a physician would utilize a tourniquet, hemostatic cotton or a hemostat to prevent profuse bleeding of a patient during a surgical procedure. But the efficiency of the hemostasis is not quite satisfying, owing to the physical restraints of the above-mentioned devices. For instance, the tourniquet is not suited for a surgery on a human chest. Therefore, a new hemostasis apparatus could serve to rectify this problem.

### SUMMARY OF THE INVENTION

An object of the present invention is to provide a hemostasis apparatus for preventing bleeding during cutaneous or subcutaneous surgeries.

Another object of the present invention is to provide a hemostasis apparatus for helping surgeries go more smoothly and safely.

A further object of the present invention is to save time and effort during surgeries.

Yet another object of the invention is to provide an economical and practical means to more easily facilitate surgery.

Other objects and advantages of the present invention will become apparent from the following descriptions, taken in connection with the accompanying drawings, wherein, by way of illustration and example, four embodiments of the present invention are disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1a is a plan view of an embodiment of the present invention;

FIG. 1b is a plan view of another embodiment of the present invention;

FIG. 2a is a plan view of another embodiment of the present invention;

FIG. 2b is a plan view of another embodiment of the present invention;

FIG. 2c is a schematic diagram illustrating how the embodiment described in FIG. 2b is applied on an arm of a patient;

FIG. 2d is a schematic diagram illustrating how another embodiment of the present invention is applied on an arm of a patient;

FIG. 3a is a plan view of another embodiment of the present invention;

FIG. 3b is a plan view of another embodiment of the present invention;

FIG. 4a is a plan view of another embodiment of the present invention;

FIG. 4b is a plan view of another embodiment of the present invention;

FIG. 4c is a schematic diagram illustrating how the embodiment described in FIG. 4b is applied on an arm of a patient;

FIG. 4d is a schematic diagram illustrating how another embodiment of the present invention is applied on an arm of a patient; and

FIG. 5 is a schematic diagram illustrating how the embodiment described in FIG. 1b is applied on a breast of a patient.

### DETAILED DESCRIPTION OF THE INVENTION

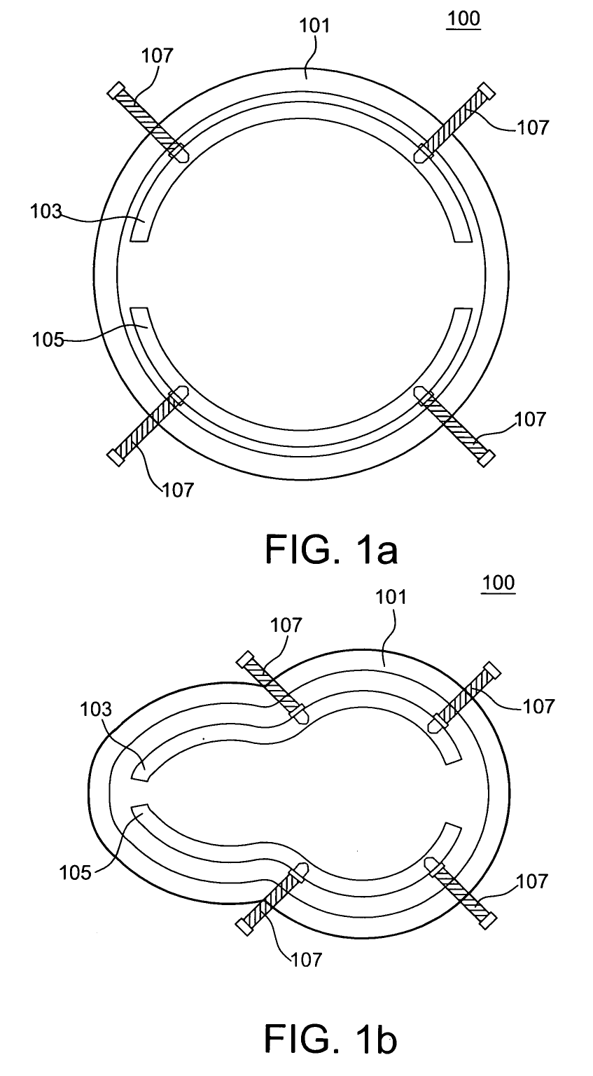

FIG. 1a is a plan view of an embodiment of the present invention. Referring now to FIG. 1a, the hemostasis apparatus 100 is used to control bleeding while performing a surgery on a surgical area of a patient. The hemostasis apparatus 100 includes a first portion 101 having a closed structure, a second portion 103 and 105, and an adjusting device 107. In this embodiment, the first portion 101 is embodied as a ring 101, the second portion 103 and 105 are embodied as two arcs 103 and 105, respectively, and the adjusting device 107 is embodied as four screws 107. The hemostasis apparatus 100 of this embodiment is preferably applied to a round surgical area of a patient, for instance, a breast. Before performing a surgical operation (either cutaneous or subcutaneous) on a breast of a patient, first the physician disposes the ring 101 on the patient to surround the breast and then affixes the ring 101 to the patient in order to maintain the relative position between the ring 101 and the patient. Second, the physician adjusts the adjusting device (or the four screws) 107 to an appropriate position to make the two arcs 103 and 105 support and stress the breast to control bleeding during the surgery. The foregoing appropriate position is determined according to different surgeries and necessities of bleeding control.

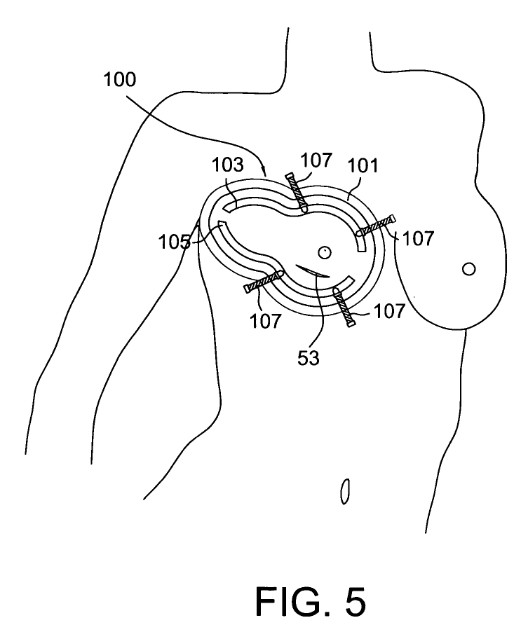

Referring to FIG. 1b, in this embodiment, the frame shape of the first portion 101 is not circular. For precisely fitting, different patients' body shapes, the shape of the first portion 101 could be customized. Referring to FIG. 5, the hemostasis apparatus 100 is applied on a breast of a patient. The shape of the first portion 101 is designed according to the patient's breast and her body shape. Therefore, the hemostasis apparatus 100 can be perfectly disposed on the surgical area of the breast and provide trustable stability between the patient and the hemostasis apparatus itself during operation. By similar way, at the outset the physician disposes the first portion 101 on the patient to surround the breast, and then affixes the first portion 101 to the patient in order to maintain the relative position between the first portion 101 and the patient. Then the physician adjusts the adjusting device (or the four screws) 107 to a appropriate position to make the second portions 103 and 105 support and stress the breast to control bleeding from a cut 53 made during the surgery.

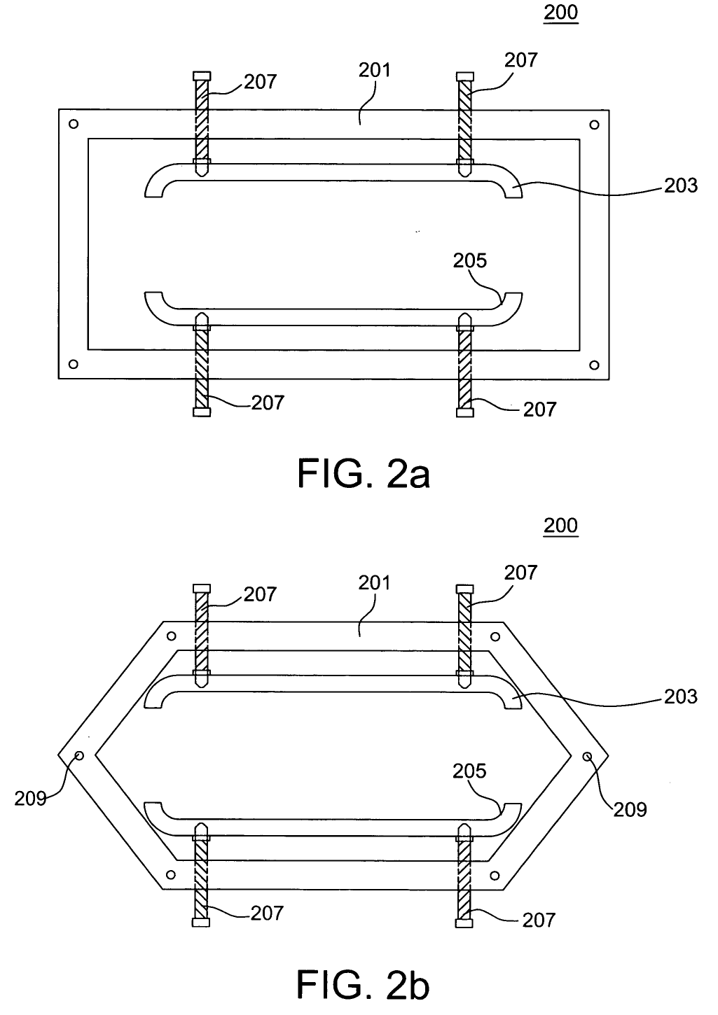

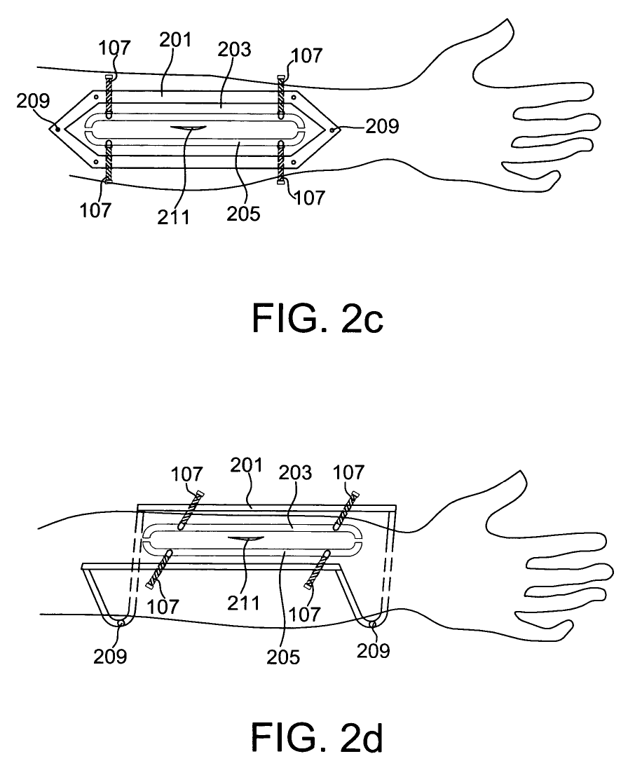

Referring now to FIG. 2a, the hemostasis apparatus 200 is used to control bleeding while performing a surgery on a surgical area of a patient. The hemostasis apparatus 200 includes a first portion 201 having a closed structure, a second portion 203 and 205, and an adjusting device 207. In this embodiment, the first portion 201 is embodied as a rectangular frame 201, the second portion 203 and 205 are embodied as two support devices 203 and 205, respectively, and the adjusting device 207 is embodied as four screws 207. The hemostasis apparatus 200 of this embodiment is preferably applied to a rectangular surgical area of a patient, for instance, an arm or a leg. Before performing a surgical operation (either cutaneous or subcutaneous) on an arm (or a leg) of a patient, the physician first disposes the rectangular frame 201 on the patient to surround the arm (or the leg), and then affixes the rectangular frame 201 to the patient in order to maintain the relative position between the rectangular frame 201 and the patient. Second, the physician adjusts the adjusting device (or the four screws) 207 to an appropriate position to make the two support devices 203 and 205 support and stress the arm (or the leg) to control bleeding during the surgery. The foregoing appropriate position is determined according to different surgeries and necessities of bleeding control.

Referring to FIG. 2b, in this embodiment, the frame shape of the first portion 201 is hexagonal rather than rectangular. For precisely fitting different patients' body shapes (or limb shapes), the shape of the first portion 201 could be customized. Referring to FIG. 2c, the hemostasis apparatus 200 is applied on an arm of a patient. The shape of the first portion 201 is designed according to the patient's arm shape. Therefore, the hemostasis apparatus 200 can be perfectly disposed on the surgical area of the arm and can provide trustable stability between the patient and the hemostasis apparatus itself during operation. By a similar method, at the outset, the physician disposes the first portion 201 on the patient to surround the surgical area of the arm, and then affixes the first portion 201 to the patient in order to maintain the relative position between the first portion 201 and the patient. It should be noticed that, the first portion 201 includes a frame-adjusting device 209, which adjusts the frame shape of the first portion 201 to help build a stable connection relationship between the first portion 201 and the arm. After the first portion 201 is arranged properly, the physician then adjusts the adjusting device (or the four screws) 207 to a appropriate position to make the second portion 203 and 205 support and stress the arm to control bleeding from a cut 211 during the surgery.

FIG. 2d shows another embodiment of the hemostasis apparatus of the present invention. The first portion 201 is a three-dimensional structure that allows an arm of a patient to pass through the first portion 201. The frame-adjusting device 209 here can also help the hemostasis apparatus 200 fit precisely on the arm of the patient. Therefore, the hemostasis apparatus 200 can be perfectly disposed on the surgical area of the arm and provide trustable stability between the patient and the hemostasis apparatus itself during operation. By a similar method, at the outset, the physician disposes the first portion 201 on the patient to surround the surgical area of the arm, and then affixes the first portion 201 to the patient in order to maintain the relative position between the first portion 201 and the patient. After the first portion 201 is arranged properly, the physician then adjusts the adjusting device (or the four screws) 207 to a appropriate position to make the second portion 203 and 205 support and stress the arm to control bleeding from a cut 211 during the surgery.

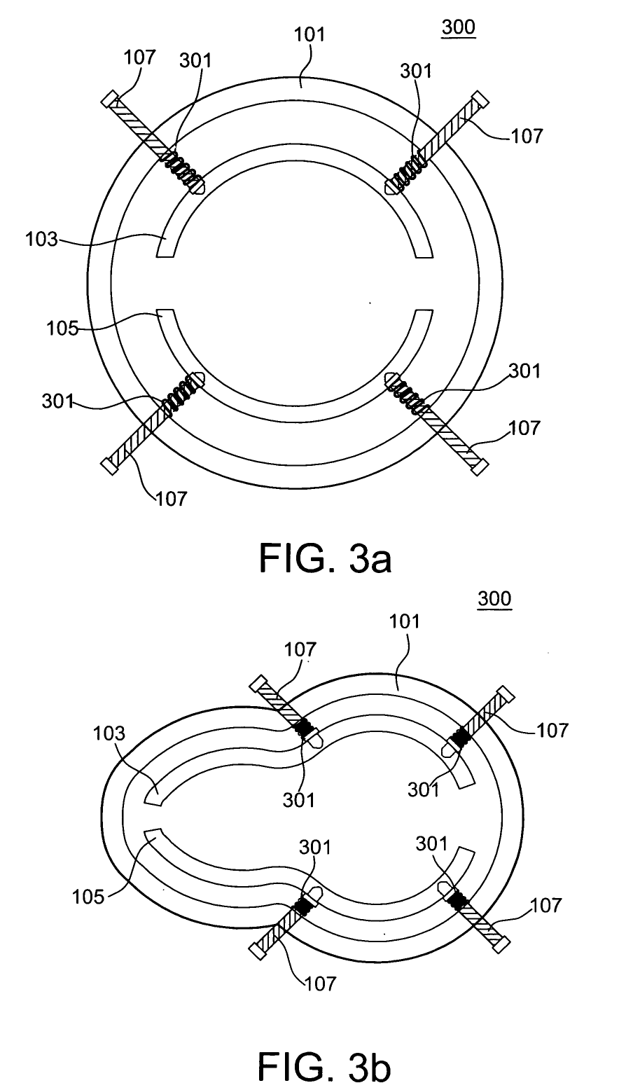

Referring now to FIG. 3a, the hemostasis apparatus 300 is used to control bleeding while performing a surgery on a surgical area of a patient. The hemostasis apparatus 300 includes a first portion 101 having a closed structure, a second portion 103 and 105, an adjusting device 107 and a resilient component 301. The resilient components 301 are connected with the adjusting device 107 to facilitate the second portion 103 and 105 to stress the patient. In this embodiment, the first portion 101 is embodied as a ring 101, the second portion 103 and 105 are embodied as two arcs 103 and 105, respectively, the adjusting device 107 is embodied as four screws 107 and the resilient component 301 is embodied as a spring 301. The hemostasis apparatus 300 of this embodiment is preferably applied to a round surgical area of a patient, for instance, a breast. Before performing a surgical operation (either cutaneous or subcutaneous) on a breast of a patient, the physician first disposes the ring 101 on the patient to surround the breast, and then affixes the ring 101 to the patient in order to maintain the relative position between the ring 101 and the patient. Second, the Physician adjusts the adjusting device (or the four screws) 107 to a appropriate position to make the two arcs 103 and 105 support and stress the breast to control bleeding during the surgery. The foregoing appropriate position is determined according to different surgeries and necessities of bleeding control. The spring 301 (or the resilient component 301) provides a force to facilitate the two arcs 103 and 105 to control bleeding during the surgery. After the surgery, the spring 301 can also provide a recovery force to facilitate the two arcs 103 and 105 reverting back to their original positions.

Referring now to FIG. 3b, the hemostasis apparatus 300 is used to control bleeding while performing a surgery on a surgical area of a patient. The hemostasis apparatus 300 includes a first portion 101 having a closed structure, a second portion 103 and 105, an adjusting device 107 and a resilient component 301. The resilient components 301 are connected with the adjusting device 107 to facilitate the second portion 103 and 105 to stress the patient. In this embodiment, the first portion 101 is embodied as a ring 101, the second portion 103 and 105 are embodied as two arcs 103 and 105, respectively, the adjusting device 107 is embodied as four screws 107 and the resilient component 301 is embodied as a spring 301. The hemostasis apparatus 300 of this embodiment is preferably applied to a round surgical area of a patient, for instance, a breast. Before performing a surgical operation (either cutaneous or subcutaneous) on a breast of a patient, the physician first disposes the ring 101 on the patient to surround the breast, and then affixes the ring 101 to the patient in order to maintain the relative position between the ring 101 and the patient. Second, the Physician adjusts the adjusting device (or the four screws) 107 to a appropriate position to make the two arcs 103 and 105 support and stress the breast to control bleeding during the surgery. The foregoing appropriate position is determined according to different surgeries and necessities of bleeding control. The spring 301 (or the resilient component 301) provides a force to facilitate the two arcs 103 and 105 to control bleeding during the surgery. After the surgery, the spring 301 can also provide a recovery force to facilitate the two arcs 103 and 105 reverting back to their original positions.

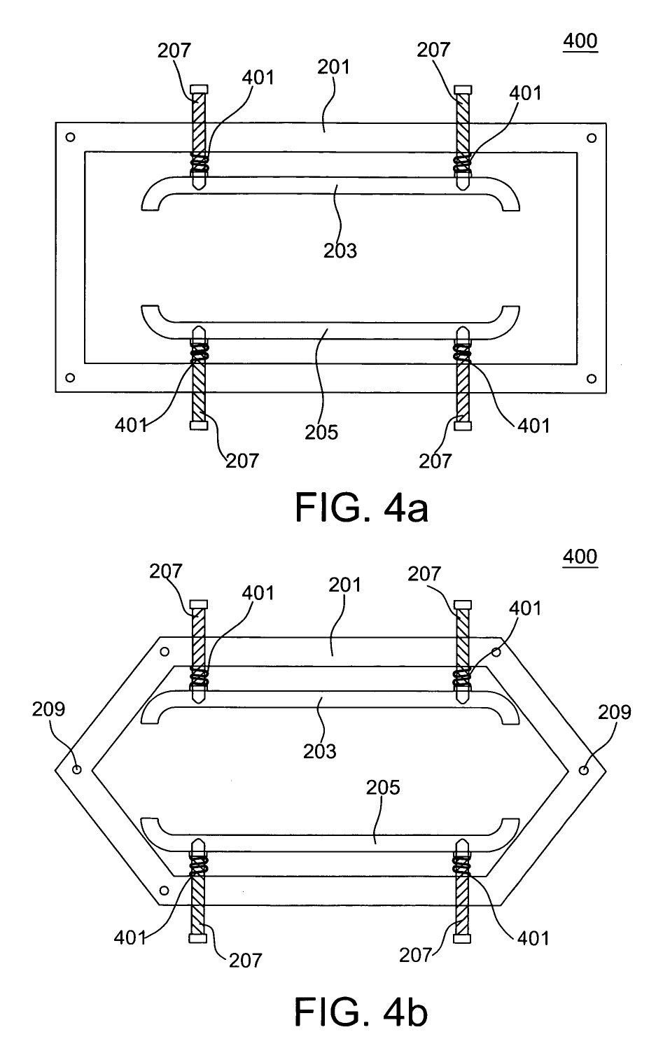

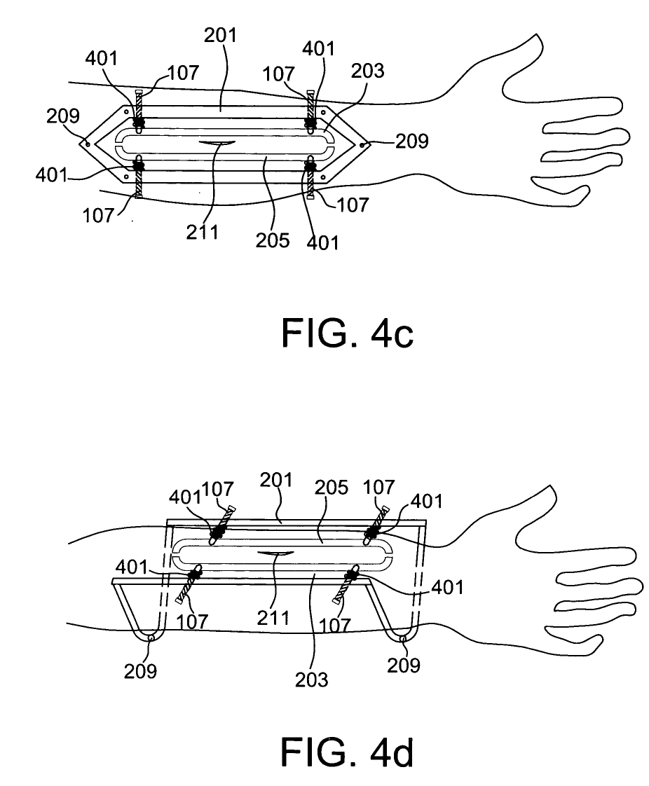

Referring now to FIG. 4a, the hemostasis apparatus 400 is used to control bleeding while performing a surgery on a surgical area of a patient. The hemostasis apparatus 400 includes a first portion 201 having a closed structure, a second portion 203 and 205, an adjusting device 207, and a resilient component 401. In this embodiment, the first portion 201 is embodied as a rectangular frame 201, the second portion 203 and 205 are embodied as two support devices 203 and 205, respectively, the adjusting device 207 is embodied as four screws 207, and the resilient component 401 is embodied as a spring 401. The hemostasis apparatus 400 of this embodiment is preferably applied to a rectangular surgical area of a patient, for instance, an arm or a leg. Before performing a surgical operation (either cutaneous or subcutaneous) on an arm (or a leg) of a patient, the physician first disposes the rectangular frame 201 on the patient to surround the arm (or the leg), and then affixes the rectangular frame 201 to the patient in order to maintain the relative position between the rectangular frame 201 and the patient. Second, the physician adjusts the adjusting device (or the four screws) 207 to a appropriate position to make the two support devices 203 and 205 support and stress the arm (or the leg) to control bleeding during the surgery. The foregoing appropriate position is determined according to different surgeries and necessities of bleeding control. The spring 401 (or the resilient component 401) provides a force to facilitate the two arcs 203 and 205 to control bleeding during the surgery. After the surgery, the spring 401 can also provide a recovery force to facilitate the support device 203 and 205 reverting back to their original positions.

FIG. 4b illustrates another embodiment of the present invention. Comparing to the embodiment described in FIG. 2b, the hemostasis apparatus 400 further includes several resilient components 401 to facilitate the second portions 203 and 205 to stress the patient to control bleeding during operation.

FIG. 4c illustrates another embodiment of the present invention. Comparing to the embodiment described in FIG. 2c, the hemostasis apparatus 400 further includes several resilient components 401 to facilitate the second portions 203 and 205 to stress the patient to control bleeding during operation.

FIG. 4d illustrates another embodiment of the present invention. Comparing to the embodiment described in FIG. 2d, the hemostasis apparatus 400 further includes several resilient components 401 to facilitate the second portions 203 and 205 to stress the patient to control bleeding during operation.

In other embodiments, the shape or structures of the first portion 101 (or 201) and the second portion 103 and 105 (or, 203 and 205) are variable, depending on the human body part that they are applied to. The first portion 101 (or 201) and the second portion 103 and 105 (or, 203 and 205) are made from materials compatible with human bodies, for example, stainless steel or rubber.

While the invention has been described in connection with what is presently considered to be the most practical and preferred embodiments, it is to be understood that the invention is not to be limited to the discovered embodiments. The invention is intended to cover various modifications and equivalent arrangements included within the spirit and scope of the appended claims.

1. A hemostasis apparatus used to control bleeding while performing a surgery on a surgical area of a patient, the hemostasis apparatus comprising:
a first portion having a closed structure;
an adjusting device connected with the first portion; and
a second portion connected with the adjusting device,
wherein the first portion is affixed to the patient to surround the surgical area, and a user adjusts the adjusting device to move the second portion to stress the patient to control bleeding during the surgery.

2. The hemostasis apparatus according to claim 1, further comprising a resilient component connected with the adjusting device used to facilitate the second portion to stress the patient.

3. The hemostasis apparatus according to claim 1, wherein the first portion is made from materials compatible with human bodies.

4. The hemostasis apparatus according to claim 1, wherein the second portion is made from materials compatible with human bodies.

5. The hemostasis apparatus according to claim 1, wherein the first portion is designed according to human body shape.

6. The hemostasis apparatus according to claim 1, wherein the first portion is a three-dimensional frame.

7. The hemostasis apparatus according to claim 1, wherein the first portion further comprises a frame-adjusting device for adjusting a frame shape of the first portion.

8. A hemostasis apparatus for use to control bleeding while performing a surgery on a surgical area of a patient with the hemostasis apparatus comprising of:
a frame having a closed structure;
an adjusting device connected with the frame; and
a support device connected with the adjusting device,
wherein the frame is affixed to the patient to surround the surgical area, and a user adjusts the adjusting device to move the support device to stress the patient to control bleeding during the surgery.

9. The hemostasis apparatus according to claim 8, further comprising a resilient component connected with the adjusting device to facilitate the support device to stress the patient.

10. The hemostasis apparatus according to claim 8, wherein the frame is a three-dimensional frame.

11. The hemostasis apparatus according to claim 8, wherein the frame is made from materials compatible with human bodies.

12. The hemostasis apparatus according to claim 8, wherein the support device is made from materials compatible with human bodies.

13. The hemostasis apparatus according to claim 8, wherein the frame is designed according to human body shape.

14. The hemostasis apparatus according to claim 8, wherein the frame further comprises a frame-adjust device for adjusting a frame shape of the frame.

15. A hemostasis method for controlling bleeding while performing a surgery on a surgical area of a patient by a hemostasis apparatus, the hemostasis apparatus includes a first portion having a closed structure, an adjusting device connected with the first portion, and a second portion connected with the adjusting device, the method comprising:
affixing the first portion to the patient to surround the surgical area; and
adjusting the adjusting device to move the second portion to stress the patient to control bleeding during the surgery.

16. The method according to claim 15, wherein the first portion is a three-dimensional frame.

17. The method according to claim 15, wherein the first portion further comprises a frame-adjust device for adjusting a frame shape of the first portion.
